(19) United States
(12) Patent Application Publication
(10) Pub. No.: US 2025/0302438 A1
(43) Pub. Date: Oct. 2, 2025

(54) FLEXIBLE DEVICE AND METHOD FOR MANUFACTURING THE FLEXIBLE DEVICE AND MONITORING SYSTEM

(71) Applicants: Felix Wu Shun WONG, Hong Kong (CN); City University of Hong Kong, Hong Kong (CN)

(72) Inventors: Bee Luan KHOO, Hong Kong (CN); Ke HUANG, Hong Kong (CN); Zhiqiang MA, Hong Kong (CN); Felix Wu Shun WONG, Hong Kong (CN)

(73) Assignees: Felix Wu Shun WONG, Hong Kong (CN); City University of Hong Kong, Hong Kong (CN)

(21) Appl. No.: 19/089,073

(22) Filed: Mar. 25, 2025

Related U.S. Application Data
(60) Provisional application No. 63/570,308, filed on Mar. 27, 2024.

(51) Int. Cl. A61B 8/00; A61B 5/391; A61B 8/02; A61B 8/08

(52) U.S. Cl. CPC A61B 8/4416; A61B 8/02; A61B 8/0866; A61B 8/565; A61B 5/391; A61B 8/4236; A61B 2562/125

(57) ABSTRACT

Embodiments of the present disclosure relate to a flexible device. The flexible device includes a flexible sensing slice. The flexible sensing slice includes: a combination of an ultrasound sensor and at least one bioelectrical sensor. The at least one bioelectrical sensor is fabricated by processing a polyimide film using a laser with Laser-Induced Graphene (LIG) as a sensing material, and the ultrasound sensor is placed on the polyimide film. The flexible sensing slice further includes: a Polydimethylsiloxane (PDMS) film encapsulating the combination of the ultrasound sensor and the at least one bioelectrical sensor.

### CROSS REFERENCE TO RELATED DOCUMENT(S)

The present application claims priority to U.S. provisional application No. 63/570,308, filed on Mar. 27, 2024, the contents of which are incorporated herein by reference in its entirety.

### TECHNICAL FIELD

Embodiments of the present disclosure generally relate to the sensing technical field, and more particularly, to a flexible device, a method for manufacturing the flexible device and a monitoring system.

### BACKGROUND

More than 200 million women undergo pregnancy each year. Despite normal pregnancy and childbirth are often uneventful, there remain unexpected ongoing risks of severe consequences in labor. Nearly all pregnant women in developed countries require safety monitoring during labor. Monitoring the vital signs of pregnant women and their fetuses in labor is therefore crucial in obstetric care to prevent fetal and maternal morbidities and mortality. There have been very limited innovations in labor monitoring systems, and the costs and unavailability of existing monitoring systems leave many women in low-income areas without this basic safety service. The traditional cardiotocography sensors often used in labor monitoring have limitations and disadvantages, such as rigidity, discomfort, and large size.

The commonly used system for monitoring fetal heart rate and uterine contractions is the cardiotocogram. It typically involves securing a rigid pressure-measuring detection unit and ultrasound Doppler with two fixed belts to the abdomen, with connecting wires to a cardiotocographic machine. These labor monitors are often bulky, expensive, inflexible, and have limited availability for other labor monitoring purposes such as premature or obstructed labor.

### SUMMARY

Embodiments of the present disclosure provide a flexible device, a method for manufacturing the flexible device and a monitoring system.

According to an aspect, there is provided a flexible device, including a flexible sensing slice, wherein the flexible sensing slice includes:
a combination of an ultrasound sensor and at least one bioelectrical sensor, wherein the at least one bioelectrical sensor is fabricated by processing a polyimide film using a laser with Laser-Induced Graphene (LIG) as a sensing material, and the ultrasound sensor is placed on the polyimide film; and
a Polydimethylsiloxane (PDMS) film encapsulating the combination of the ultrasound sensor and the at least one bioelectrical sensor.

According to another aspect, there is provided a method for manufacturing a flexible device, including:
fabricating at least one bioelectrical sensor by processing a polyimide film using a laser with Laser-Induced Graphene (LIG) as a sensing material, and reserving a space for an ultrasound sensor;
placing the ultrasound sensor in the space; and
placing a PDMS film to encapsulating the combination of the ultrasound sensor and the at least one bioelectrical sensor.

According to another aspect, there is provided a monitoring system including:
a flexible device, including a flexible sensing slice,
wherein the flexible sensing slice includes:
a combination of an ultrasound sensor and at least one bioelectrical sensor,
wherein the at least one bioelectrical sensor is fabricated by processing a polyimide film using a laser with Laser-Induced Graphene (LIG) as a sensing material, and the ultrasound sensor is placed on the polyimide film; and
a PDMS film encapsulating the combination of the ultrasound sensor and the at least one bioelectrical sensor; and
a terminal configured to receive signals of the ultrasound sensor and the at least one bioelectrical sensor wirelessly.

### BRIEF DESCRIPTION OF THE DRAWINGS

The accompanying drawings, which are incorporated herein and form a part of the specification, illustrate embodiments of the present disclosure and, together with the description, further serve to explain the principles of the present disclosure and to enable a person skilled in the pertinent art to make and use the present disclosure.

FIG. 1 is a schematic diagram of a flexible device according to an embodiment of the present disclosure.

FIG. 2 is a schematic construction illustration of a flexible sensing slice in the flexible device.

FIG. 3 is a schematic diagram of an example flexible sensing slice according to an embodiment of the present disclosure.

FIG. 4 is a schematic diagram of a flexible device according to an embodiment of the present disclosure.

FIG. 5 is a schematic construction illustration of a controller in the flexible device according to an embodiment of the present disclosure.

FIG. 6 is a schematic diagram of a monitoring system according to an embodiment of the present disclosure.

FIG. 7 is a schematic diagram of a monitoring system according to an embodiment of the present disclosure.

FIG. 8 is a schematic diagram showing a workflow for the monitoring system.

FIG. 9A and FIG. 9B illustrate the principle of ultrasonic transducer wafer according to an example of the present disclosure.

FIG. 10 is a schematic flowchart of a method for manufacturing a flexible device according to an embodiment of the present disclosure.

FIG. 11 illustrates an example of a fabrication process according to an embodiment of the present disclosure.

FIG. 12 shows existing cardiotocograph sensors for monitoring the labor process.

### DETAILED DESCRIPTION

Although specific configurations and arrangements are discussed, it should be understood that this is done for illustrative purposes only. A person skilled in the pertinent art will recognize that other configurations and arrangements can be used without departing from the spirit and scope of the present disclosure. It will be apparent to a person skilled in the pertinent art that the present disclosure can also be employed in a variety of other applications.

It is noted that references in the specification to “one embodiment,” “an embodiment,” “an example embodiment,” “some embodiments,” “certain embodiments,” etc., indicate that the embodiment described may include a particular feature, structure, or characteristic, but every embodiment may not necessarily include the particular feature, structure, or characteristic. Moreover, such phrases do not necessarily refer to the same embodiment. Further, when a particular feature, structure, or characteristic is described in connection with an embodiment, it would be within the knowledge of a person skilled in the pertinent art to affect such feature, structure, or characteristic in connection with other embodiments whether or not explicitly described.

In general, terminology may be understood at least in part from usage in context. For example, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

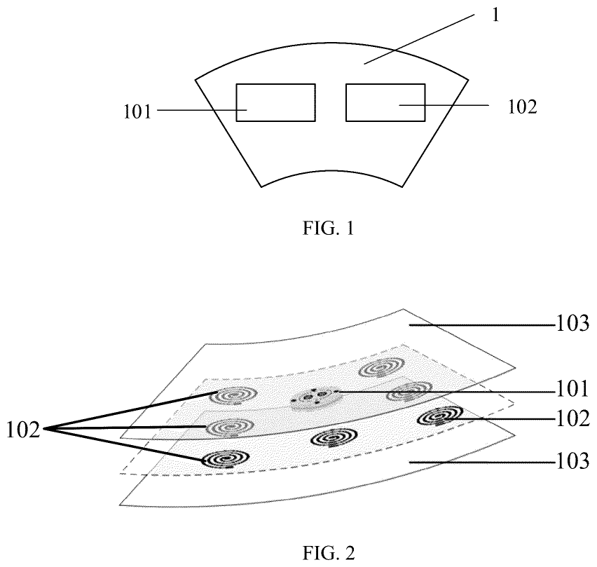

FIG. 1 is a schematic diagram of a flexible device according to an embodiment of the present disclosure. FIG. 2 is a schematic construction illustration of a flexible sensing slice in the flexible device. Referring to FIG. 1 and FIG. 2, the flexible device includes a flexible sensing slice 1. The flexible sensing slice 1 includes a combination of an ultrasound sensor 101 and at least one bioelectrical sensor 102. The at least one bioelectrical sensor 102 is fabricated by processing a polyimide film using a laser with Laser-Induced Graphene (LIG) as a sensing material, and the ultrasound sensor 101 is placed on the polyimide film. A Polydimethylsiloxane (PDMS) film 103 (e.g., top PDMS encapsulation film, bottom PDMS encapsulation film) encapsulates the combination of the ultrasound sensor 101 and the at least one bioelectrical sensor 102 (see FIG. 2).

In the flexible device, the flexible sensing slice is fabricated by processing a polyimide film using a laser with Laser-Induced Graphene (LIG) as a sensing material. Thus, the flexible sensing slice has ultra-thin and flexible nature. The flexible sensing slice can be affixed directly to a pregnant woman's abdomen, thus ensuring ease of use and comfort.

Further, the PDMS film encapsulates the combination of the ultrasound sensor and the bioelectrical sensor(s), and thus the PDMS film can protect the sensors, reduces external interference, and improves sensitivity. The PDMS film also makes the sensors flexible, so that the sensitivity can be enhanced by lowering sensor rigidity, making it more fit for human skin. That is to say, the flexible device (or called sensor chip) includes flexible housing materials, laser-induced graphene and Polydimethylsiloxane (PDMS) that protect the sensor chip from environmental impacts and ensure comfort and durability.

In addition, this embodiment proposes a novel combination of sensors (bioelectrical sensor(s) and ultrasound sensor) to monitor, e.g., the uterine labor process and the baby's well-being.

According to an embodiment, the bioelectrical sensor(s) includes multiple bioelectrical sensors in an array of rows and columns for measuring uterine contraction indexes, e.g., including frequency, intensity, propagation velocity, and movement direction. The ultrasound sensor includes an ultrasonic transducer configured to measure a fetal heart rate.

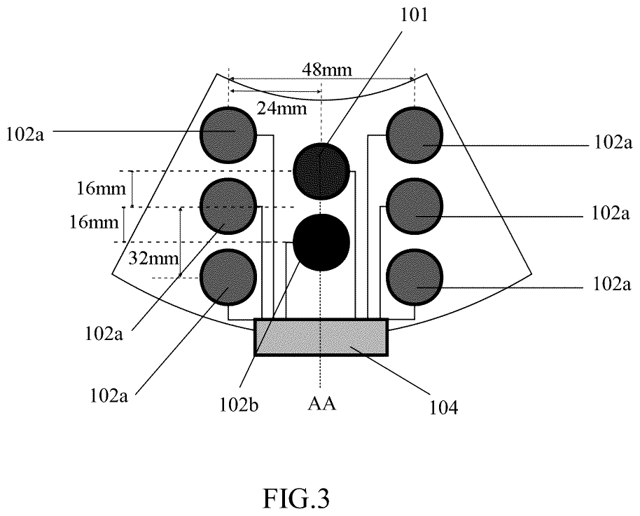

FIG. 3 is a schematic diagram of an example flexible sensing slice according to an embodiment of the present disclosure. The flexible sensing slice may be a flexible membrane slice. The at least one bioelectrical sensor includes six electrohysterography (EHG) electrodes 102a arranged in three rows and two columns for EHG signal reading and one reference EHG electrode 102b for reference measurement. The one reference EHG electrode 102b is arranged between two columns of the six EHG electrodes. For example, the two columns may be arranged symmetrically with respect to a virtual line (e.g., the dotted line AA in FIG. 3) for connecting a center of the reference EHG electrode 102b and a center of the ultrasound sensor 101.

The three-row and two-column electrode layout offers several key advantages over other electrode configurations, such as ring or fan layout, regarding detection and measurement capability. These advantages are outlined as follows:

### 1. Signal Stability

The three-row and two-column electrode layout effectively capture signals from various directions, ensuring uniform signal acquisition. This configuration reduces the instability typically caused by poor electrode placement, a common issue in ring or fan layout. In contrast, ring and fan layouts can result in weaker signal reception in specific directions, leading to inaccurate or incomplete data, especially in areas where the signal coverage is not optimal.

### 2. Detection of Contraction Direction

The three-row and two-column electrode layout enables the arrangement of electrodes to cover different contraction directions effectively. This design is crucial for accurately detecting the direction of contractions, which is vital in obstetric monitoring. By capturing directional information, this configuration helps to distinguish between effective and ineffective contractions, ensuring a more precise labor assessment. In contrast, ring or fan layout may struggle to detect the full range of contraction directions, leading to potential gaps in data.

### 3. Noise Suppression

The three-row and two-column electrode layout uses a differential signal acquisition approach, where adjacent electrodes compare signals to minimize the impact of external noise and interference. This method enhances the clarity and accuracy of the captured signals. In contrast, a ring layout is more susceptible to environmental noise, as signals in specific directions may be more affected by interference, leading to a degradation in signal quality.

### 4. Strong Adaptability

One of the significant advantages of the three-row and two-column electrode layout is its adaptability. This configuration can be easily adjusted based on the specific monitoring needs and environmental conditions. For instance, the number of electrodes in the rows or columns can be modified based on variations in the test results, providing a flexible solution that other layouts, like the ring or fan layout, may not offer to the same extent.

### 5. Easier Signal Processing

The structured arrangement of electrodes in three rows and two columns simplifies the data processing and algorithm implementation. The consistent nature of the electrode signals facilitates the application of machine learning and data analysis algorithms for signal classification and anomaly detection. This streamlined process is especially beneficial in complex signal environments, making identifying meaningful patterns and anomalies easier, which may be harder to detect with the more scattered or less structured signals from a ring or fan layout.

### 6. Simplified Design

Compared to the other layout such as a ring layout, the three-row and two-column design is simpler and more intuitive. It allows for a higher concentration of electrodes in specific areas, which improves the sensor's sensitivity and overall detection capability. The simplified design also reduces the system's manufacturing and usage complexity, making it more efficient and cost-effective.

The primary function of the reference EHG electrode is to serve as a baseline or reference point for the electrical signals captured by the surrounding active electrodes. While its role is not solely focused on noise reduction, the reference electrode is crucial in minimizing the effects of common noise and interference. Providing a stable baseline or zero point for signal measurements helps ensure that the recorded signals are accurate and consistent. The reference electrode allows for the subtraction of noise or interference that affects both the reference and the active electrodes, enhancing the signal quality during the processing stage. In this way, the reference electrode contributes to the overall signal integrity, mitigating unwanted noise. Additionally, in specific designs, the reference electrode may also function as a grounding point, stabilizing the system electrically and further reducing the risk of signal distortion caused by electrical interference.

The position of the reference EHG electrode is vital for optimal signal acquisition and measurement consistency. Ideally, the reference electrode should be placed along the symmetry line of the two columns of EHG electrodes. This placement ensures that the signal capture from both sides of the abdomen is balanced, which is critical for accurate and uniform readings of uterine contractions. The symmetry line positioning improves the consistency of measurements across the array of active electrodes, as it minimizes discrepancies that could arise from uneven signal acquisition. Positioning the reference electrode in this way helps achieve more accurate and reliable assessments of electrical activity. Although the reference electrode can technically be placed elsewhere, positioning it along the symmetry line is the most effective way to enhance the quality of the signal and improve the accuracy of contraction measurements.

As shown in FIG. 3, the vertical spacing between adjacent EHG electrodes 102a among the six EHG electrodes 102a may be equal. For example, the vertical spacing between adjacent EHG electrodes 102a may be equal to 32 mm. The vertical spacing between the center of the ultrasound sensor 101 and the center of an adjacent EHG electrode 102a may be 16 mm, and the vertical spacing between the center of the reference electrode 101 and the center of an adjacent EHG electrode may be 16 mm. The EHG signals array can measure the value, frequency, propagation velocity, and movement direction of the uterine myoelectric activity in the abdominal region and process the signals through AI-assisted algorithms to judge the labor status. The ultrasound sensor 101 may also be called a FHR sensor.

The spacing of 32 mm between adjacent rows among the three rows of electrodes is a carefully designed feature that plays a crucial role in accurately extracting the direction of uterine contractions and screening for invalid contractions. This specific distance is chosen to optimize spatial resolution, signal differentiation, and the ability to analyze contraction directionality effectively.

### Effectively Extracting the Direction of Contractions

The 32 mm spacing between the adjacent rows among three rows of electrodes is significant because it allows the system to capture the electrical activity generated by uterine contractions with optimal differentiation. This spacing is key to distinguishing the direction the contraction wave propagates through the uterine tissue.
Optimal Signal Differentiation: The spacing of 32 mm ensures that the electrodes are neither too close nor too far apart, which is essential for capturing clear and distinct signals from different contraction locations within the uterus. If the electrodes are too close, the electrical signals from adjacent contraction sites may overlap, making it challenging to analyze the propagation direction. On the other hand, if the electrodes are too far apart, the system may miss localized or early-stage contractions, reducing the system's ability to detect and interpret the contraction dynamics accurately. The 32 mm spacing strikes the right balance for capturing electrical signals from multiple points within the uterine tissue and effectively triangulates the contraction wave's direction.
Triangulation of Signals: By strategically spacing the electrodes 32 mm apart, the system can use differences in signal strength and timing between the rows to triangulate the direction in which the contraction propagates. This is possible because the signals from each row are slightly offset, allowing the system to compare the timing of the electrical activity detected at each row and determine the direction of the contraction wave. This capability is crucial for distinguishing between effective and ineffective contractions.

### Timely Screening for Invalid Contractions

The 32 mm spacing also enhances the system's ability to promptly identify and classify invalid contractions, contributing to the accuracy and reliability of the contraction monitoring.
Differentiation of Valid and Invalid Contractions: The configuration with 32 mm spacing effectively compares signals across the three rows. When a contraction occurs, it produces a distinct pattern of electrical activity, which propagates through the uterine muscle. If the contraction is valid and adequately propagating, the system will detect a consistent pattern of increasing signal strength as the contraction wave moves across the rows. If the signals are weak or inconsistent across the rows or there is no clear propagation direction, the system can flag the contraction as invalid. The 32 mm spacing ensures that the system has enough spatial resolution to differentiate between legitimate contractions and those that may be artifacts or weak signals that are not meaningful in the context of labor.
Timely Detection of Signal Inconsistencies: Because the electrodes are spaced 32 mm apart, the system can quickly analyze the pattern and timing of the signals across the three rows, quickly identifying invalid contractions. For instance, if a contraction is detected in one row but not in the adjacent rows or if the contraction pattern is inconsistent with expected behaviors (e.g., weak or scattered signals), the system can promptly classify it as invalid. This is crucial in clinical settings, where timely and accurate decision-making is essential for ensuring maternal and fetal safety.

The choice of 32 mm for the distance between adjacent rows is based on the need for a balance between spatial resolution and signal capture accuracy. This distance allows the system to:
Capture electrical activity at the correct resolution: It is within the optimal range (typically 30 mm to 40 mm) to detect and analyze contraction signals effectively.
Differentiate between contractions: It ensures the system can distinguish between signals from multiple contraction sites, allowing for precise identification of the direction of contraction propagation.
Minimize signal overlap: It avoids the issue of signal overlap, which would occur if the electrodes were too close together, while still providing sufficient coverage to capture localized contraction activity.

As shown in FIG. 3, the flexible sensing slice is an ultra-thin, conformal, and flexible multi-pregnancy parameter sensing slice for Safe Labor Monitoring (SLM). It is designed to monitor fetal heart rate and uterine contraction patterns (frequency, intensity, propagation velocity, and movement direction) throughout labor. The flexible sensing slice enables effective clinical supervision of laboring women's vital signs and fetuses. The flexible sensing slice can adhere to the curved skin surface autonomously, enabling the detection and effective management of safety indicators in a comfortable manner that is both user-friendly for pregnant women and safe for the fetus.

That is to say, the present disclosure provides a multi-mode flexible wireless sensor for detecting pregnant women's fetal heart rate and uterine contraction indexes, including frequency, intensity, propagation velocity, and movement direction. The flexible wireless sensor may also be considered as a flexible and self-attaching sensor chip with multiple sensitive elements, including seven EHG sensing units and one ultrasonic transducer unit, which can be fitted closely to the pregnant woman's skin to improve measurement accuracy.

The flexible sensing slice (or called a sensing module) is positioned directly beneath the umbilicus of a pregnant woman, which integrates an onboard ultrasound sensor, such as a wireless Doppler ultrasound sensing unit, and an electrohysterography (EHG) sensing unit, e.g., including six EHG electrodes and one reference EHG electrode. The EHG sensor (or called EHG electrode) is fabricated using laser-induced graphene (LIG) as the sensing material. LIG is prepared through laser ablation of commercial polyimide films, offering several advantages, such as a porous structure, cost-effectiveness, high yield, and exceptional sensitivity. The sensing slice has the characteristics of ultra-thin and ultra-flexible self-adhesive, which can be completely attached to the lower side of the belly button of pregnant women without external force. It incorporates multiple sensing units to ensure the stability of the sensing signal, enabling multi-directional and high-precision monitoring of abnormal signals.

As shown in FIG. 3, the flexible sensing slice further includes a first Serial Peripheral Interface (SPI) component 104. The first SPI component 104 is connected with the ultrasound sensor 101 and the bioelectrical sensors 102a and 102b. The first SPI component can be detachably connected with a second SPI component in a controller which will be described later.

The underlying SPI design enables convenient disassembly and replacement of the flexible sensing slice, ensuring a more discreet and hygienic usage experience while minimizing the risk of disease transmission in public healthcare settings.

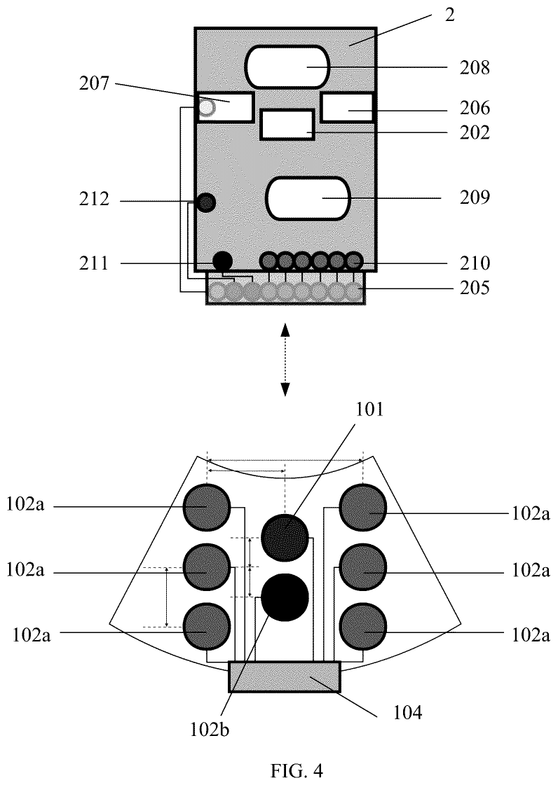

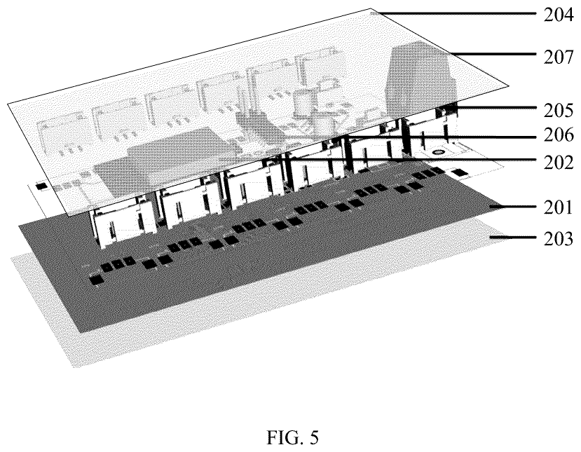

FIG. 4 is a schematic diagram of a flexible device according to an embodiment of the present disclosure. FIG. 5 is a schematic construction illustration of a controller in the flexible device according to an embodiment of the present disclosure. As shown in FIG. 4, the flexible device includes the flexible sensing slice 1 and further includes a controller 2. The controller 2 is configured to receive and process signals of the ultrasound sensor 101 and the bioelectrical sensors 102. As shown in FIG. 5, the controller 2 includes a flexible printed circuit board 201 and a microcontroller unit (MCU) 202 formed on the flexible printed circuit board. The controller 2 further includes top and bottom PDMS films 203 and 204 (or called top and bottom encapsulation films) encapsulating the flexible circuit board 201. The MCU 202 may be low-power microcontroller unit on the controller for processing and analyzing measurement data in real-time and controlling power consumption during transmission.

In the controller 2, the MCU 202 is formed on the flexible printed circuit board 201. On the one hand, the MCU can process and analyze measurement data (the signals of the ultrasound sensor 101 and the bioelectrical sensors 102) in real-time. On the other hand, the controller 2 can also be attached to curved skin surface, enabling conformal monitoring.

As shown in FIG. 4, the controller 2 may further include a second SPI component 205. The second SPI component 205 can be detachably connected with the first SPI component 104 in the flexible sensing slice 1.

According to an embodiment, the controller 2 may further include a transceiver 206 and a flexible battery 207. The transceiver 206 is configured to transmit measurement data of the ultrasound sensor and the bioelectrical sensor(s) to a terminal wirelessly. For example, the transceiver 206 may be a wireless transceiver that uses Bluetooth technology and can be easily connected to various mobile devices, such as smartphones and computers. The flexible battery 207 is connected with the second SPI component 205 to provide power supply for the flexible sensing slice. The power supply of the flexible device (i.e., the sensor chip) is supplied by the flexible battery that can meet the needs of long-term use (can be used for 6-10 hours per charge). The transceiver and the flexible battery are printed on the flexible printed circuit board.

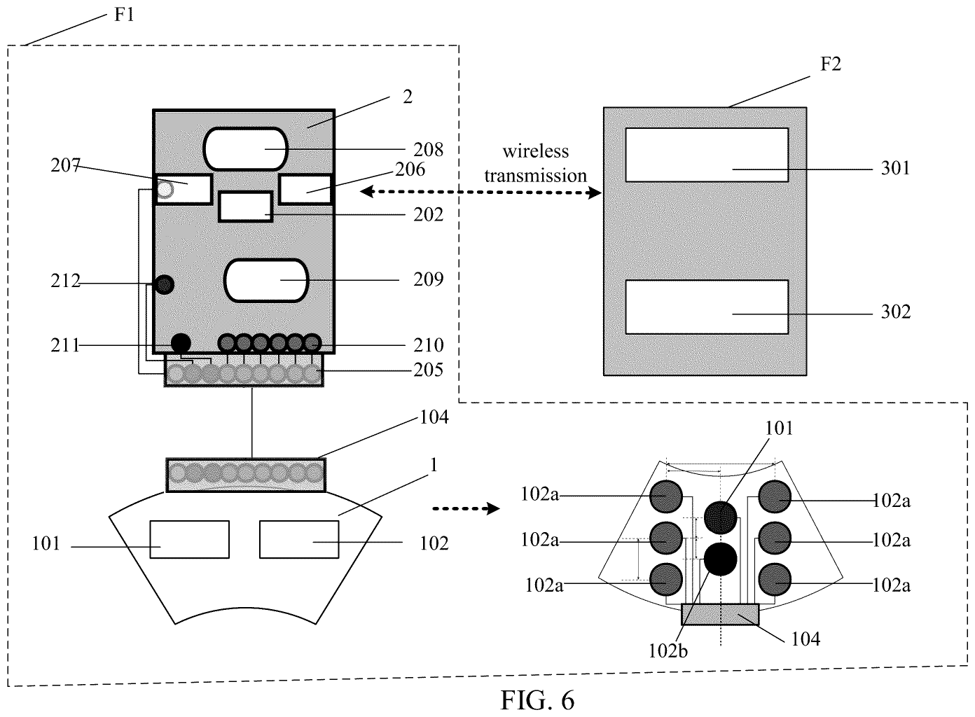

FIG. 6 is a schematic diagram of a monitoring system according to an embodiment of the present disclosure. As shown in FIG. 6, the monitoring system includes the flexible device F1 and a terminal F2. The terminal F2 may be a base station or a mobile device (e.g., a laptop, or a microblaze). The terminal F2 is configured to receive measurement data of the ultrasound sensor and the bioelectrical sensor(s) wirelessly.

The terminal F2 may include a data processing system 301 and a visual interface 302. The data processing system 301 may be configured to analyze measurement data of the ultrasound sensor and the bioelectrical sensor(s). Specifically, the data processing system may be configured to receive and analyze the data from the sensor chip and provide real-time monitoring and analysis results. The data processing system may embed a data processing model, e.g., an embed signal processing model. The visual interface 302 displays various physiological curves of fetal heart patterns, or uterine contractions based on analysis result(s) of the data processing system. The visual interface 302 may further display health evaluation results to users through an artificial intelligence (AI) auxiliary system.

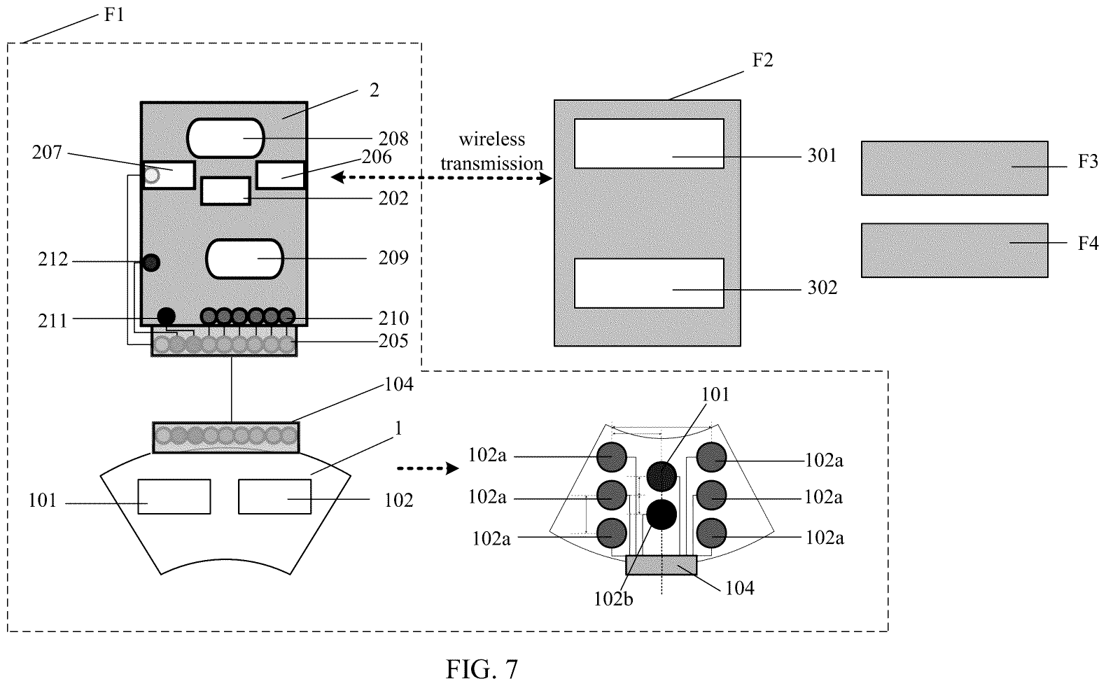

FIG. 7 is a schematic diagram of a monitoring system according to an embodiment of the present disclosure. As shown in FIG. 7, the monitoring system further includes a cloud server F3 and an alarm (or called an alarm module) F4. The cloud server F3 is configured to uploading data to a cloud for data storage and analysis. The alarm F4 is configured to send an alarm signal when an abnormal situation is detected, e.g., based on analysis of the data processing system. By sending the alarm signal, action can be taken timely.

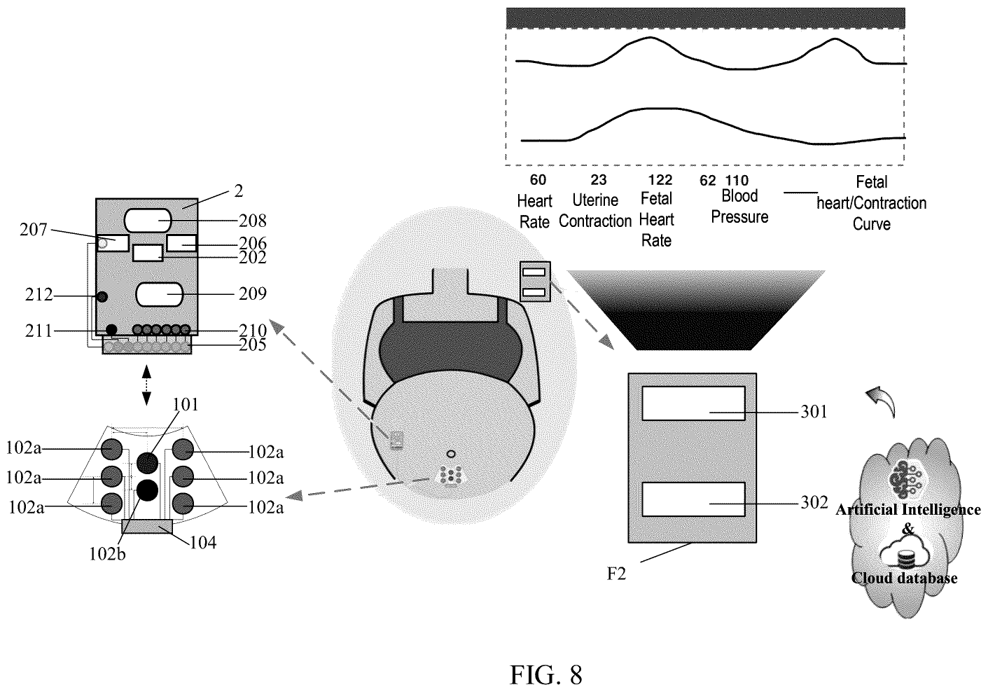

FIG. 8 is a schematic diagram showing a workflow for the monitoring system. The monitoring system may be used as a labor cardiotocogram monitoring system used in public medical units and hospitals. The labor cardiotocogram monitoring system requires frequent repetitive usage, which is costly to repair for wear and tear and lack effective cleaning methods for contaminations. In the embodiments of the present disclosure, the flexible sensing slice 1 and the controller 2 are connected via the first SPI component 104 and the second SPI component 205. The controller 2 provides the power supply for the flexible sensing slice 1. Thus, each sensing slice can be disposable after use. This design is environmentally friendly and cost-effective for disassembling and replacing the sensing slice (or called a sensor slice).

The controller 2 includes the flexible battery 207 which may be a rechargeable power module, and a microcontroller unit (MCU) (or called a microcontroller chip) 202. Further, the controller 2 may incorporate an amplifier or a filter 208, a converter 209 (e.g., an ADC, analog-to-digital converter), and a transceiver 206 (e.g., a Bluetooth module) shown in FIG. 4, and all of them can work successfully under the control of the MCU 202. Bluetooth connects the controller via the serial port peripheral interface and sensing module to transmit the preliminary multi-mode signal to the terminal. These components may be all printed on a Flexible Printed Circuit Board (FPCB) and packaged between the top and bottom PDMS encapsulation. The controller 2 may also be provided with EHG inputs 210 (for receiving signals of the EHG electrodes), a reference input 211 (for receiving a signal of the reference EHG electrode) and an FHR input 212 (i.e., input for receiving a signal of the ultrasound transducer) for receiving corresponding signals from the second SPI component 205.

The terminal includes the data processing system (or called as signal processing system) and the visual interface (such as Graphical User Interface (GUI)). The terminal may employ a proposed artificial intelligence algorithm to analyze the signal(s) or measurement data in real-time on the visual interface, displaying fetal heart rate/contraction curves and various wearer indicator parameters while assessing their health level using an AI-assisted system.

The monitoring system offers an onboard wireless Doppler ultrasound (US) and EHG sampled 500 Hz. The sensing slice is placed below the participant's belly button to derive FHR (fetal heart rate) via ultrasound Doppler and uterine contraction via EHG. The underside of the sensing slice exposes seven laser-induced graphene electrodes and an ultrasonic transducer, which faces the skin (see FIG. 2). The ultrasonic transducer converts high-frequency electrical energy into mechanical energy.

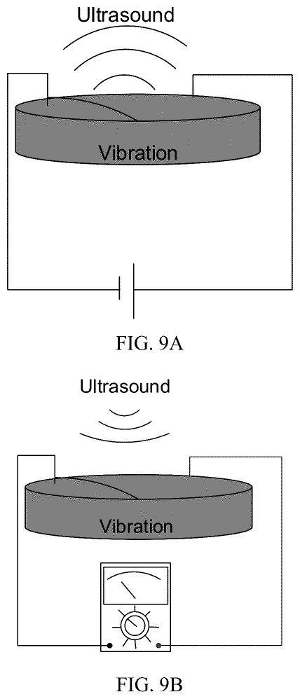

When the ultrasonic ceramic plate is used as an ultrasonic generator, Alternating Current (AC) voltage needs to be applied. When a voltage is applied to the piezoelectric ceramic plate, the shape of the ceramic plate will change due to the piezoelectric effect. This shape change involves compression and expansion. This rapid expansion and compression of the ceramic sheet produces vibrations, which propagate into the medium through ultrasonic waves, as shown in FIG. 9A. When the external ultrasonic wave hits the ceramic piece, the shape of the ceramic piece will be changed by the force. Due to the inverse piezoelectric effect, this change in the shape of the ceramic sheet creates a voltage that can be measured and converted into an electrical signal, as shown in FIG. 9B.

Referring to FIG. 3, the sensing slice has six EHG electrodes for EHG signal readings and one for reference measurements. FIG. 3 is provided for illustration only, and the configuration can be changed depending on the test result. The six reading electrodes are distributed in three rows and two columns, and the distribution spacing of the three rows of electrodes can effectively extract the direction of contraction and timely screen for invalid contraction. The left and right electrodes in the same row can make the signal more stable in intensity and frequency, making the analysis results more reliable. They can also be used as bases for judging the direction of contraction.

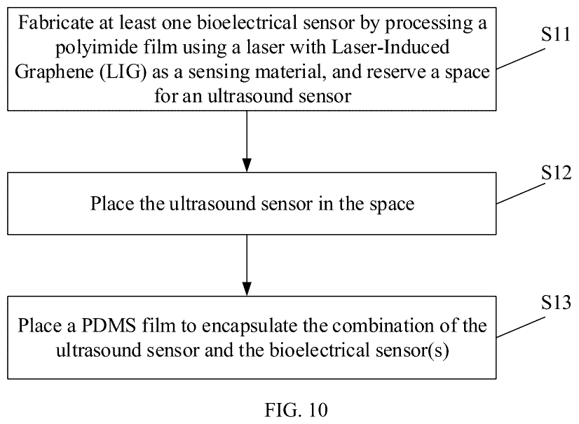

FIG. 10 is a schematic flowchart of a method for manufacturing a flexible device according to an embodiment of the present disclosure. The method includes the following steps:

In step S11, at least one bioelectrical sensor is fabricated by processing a polyimide film using a laser with Laser-Induced Graphene (LIG) as a sensing material, and a space for an ultrasound sensor is reserved.

In step S12, the ultrasound sensor is placed in the space.

In step S13, a PDMS film is placed to encapsulate the combination of the ultrasound sensor and the at least one bioelectrical sensor.

Before placing the ultrasound sensor in the space, the method further includes:
pouring PDMS liquid on the polyimide film on which pattern(s) of the bioelectrical sensor(s) has been completed;
exposing the polyimide film with the PDMS liquid poured; and
removing a PDMS layer from the polyimide film after being heated by a hot drying plate.

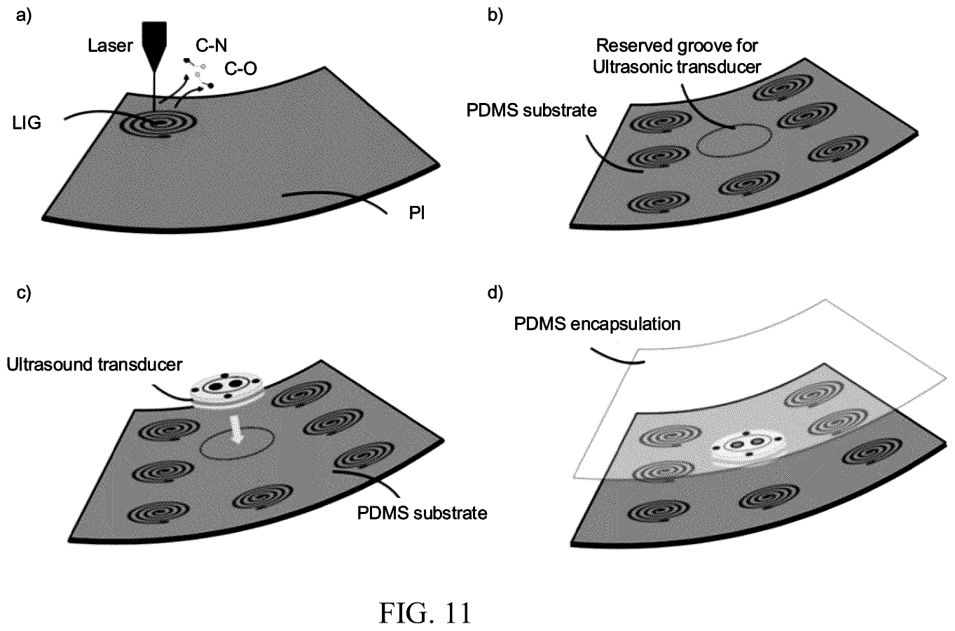

FIG. 11 illustrates an example of a fabrication process according to an embodiment of the present disclosure. The method described is for illustrative purposes only. Variations or other proper refining processes may also be possible.

The embodiment uses laser-induced graphene materials similar to other sensors. The LIG sensing unit is fabricated by laser processing commercial polyimide film(s) for easy mass production capabilities. As shown in FIG. 11:

1. First, the 3D LIG structure is prepared on a commercial PI film using a CO₂ laser, reserving the space for the ultrasonic transducer as shown in a) of FIG. 11.

2. PDMS liquid is poured on the patterned completed sample and exposed to air for about 15 minutes. The PDMS layer is removed from the PI sheet after being heated by a hot drying plate at 80° C. for 1 hour, as shown in b) of FIG. 11.

3. The ultrasound transducer is placed in the reserved position (for example, reserved groove for the ultrasonic transducer) in the PDMS substrate groove shown in c) of FIG. 11.

4. An ultra-thin PDMS film about 40 μm thick is spun on the device as the packaging layer shown in d) of FIG. 11.

Finally, a total of seven EHG electrodes are located on the sensing slice uniformly: six for channel readings and one for reference measurement in this stage (will be changed depending on the test result), as shown in FIG. 3. For each EHG electrode, the distinctive ring design maximizes the sensing area while providing enhanced flexibility and strain capacity for optimal body conformity.

The flexible sensing slice manufactured according to the above method may be self-attaching. The “self-attaching” property of the PI film may be primarily achieved through a combination of the material's inherent properties and surface treatments. Polyimide (PI) film has the potential for self-adhesion due to its ability to interact with the skin without requiring an additional adhesive layer. This is because the film can be treated or modified to enhance its surface energy, which increases its bonding capability with the skin.

To achieve this, the surface of the PI film can undergo treatments such as plasma treatment or chemical modifications. These treatments increase the film's surface energy, improving its wettability and promoting better adhesion to the skin. Altering the surface characteristics of the PI film enables the film to adhere more effectively without the need for a separate adhesive layer.

Additionally, while the self-adhesion effect can be enhanced by surface treatments, in some designs, an adhesive may be applied to the back of the PI film to increase its attachment strength to the skin further. This adhesive layer works with the surface modifications to provide a reliable bond, ensuring the film stays in place during use.

According to an embodiment, the method for manufacturing the flexible device may further includes: forming a flexible printed circuit board, wherein a microcontroller unit (MCU) is formed on the flexible printed circuit board; and placing top and bottom PDMS films to encapsulating the flexible printed circuit board.

According to an embodiment, forming the flexible printed circuit board includes:
coating a conductive material on a flexible substrate;
applying a photoresist material on the flexible substrate, performing exposure to ultraviolet (UV) light through a photomask containing a desired circuit pattern;
developing exposed photoresist material, and leaving a patterned resist layer that protects underlying conductive traces formed from the conductive material; and
attaching the MCU to the flexible substrate; and
coating a protective layer on the flexible substrate.

The following gives an example for the fabrication of the controller:

1. Design and Layout: The flexible circuit design may be created using computer-aided design (CAD) software. The circuit layout is optimized to ensure proper functionality and reliability in a flexible form factor. Components are carefully placed and connected with traces and vias to form the desired circuit topology.

2. Substrate Selection and Preparation: The flexible substrate is polyimide film based on its electrical and mechanical properties and compatibility with the desired circuit manufacturing processes. The conductive layer is copper foil, and the insulating layer is polyimide film. The chosen substrate is thoroughly cleaned using solvents to remove any contaminants or residues that could affect circuit performance.

3. Substrate Coating and Etching: The cleaned flexible substrate is coated with a thin layer of conductive material (Cu) using Physical Vapor Deposition (PVD) or electroless plating techniques. A photoresist material is applied to the copper-coated substrate, followed by exposure to ultraviolet (UV) light through a photomask containing the desired circuit pattern. The exposed photoresist is developed, leaving behind a patterned resist layer that protects the underlying copper traces.

4. Component Attachment: Electronic components, such as MCU, resistors, capacitors, and BLE SoC (Bluetooth low energy system on chip), are then attached to the flexible substrate using surface mount technology (SMT), as shown in FIG. 5. Components with solder pads are placed onto the substrate and reflow soldering is performed using a temperature-controlled oven or a reflow soldering machine. Conductive adhesives or specialized bonding techniques like flip-chip bonding are utilized for components without solder pads.

5. To ensure long-term reliability and protection against environmental factors, the fabricated flexible circuit is coated with a protective layer (silicone).

In view of the above, one or more embodiments include a portable integrated sensor system specifically designed for easy application to monitor fetal heart rate and uterine contraction strength for women in labor. An ultra-thin sensor design and self-adhesive capability significantly enhance monitoring comfort while ensuring the material's composition and wearing method are user-friendly. One or more embodiments offer a cost-effective solution by introducing a unique design to renew the electrode module, ensuring the monitoring equipment's cleanliness and privacy. By integrating wireless electronic technology and artificial intelligence-assisted diagnosis of digital data, this innovation enables the multi-indicator clinical study to assess the health status of mother and fetus in labor and provide timely warnings for potential labor risks.

One or more embodiments propose a non-invasive, ultra-thin, portable, and flexible multi-parameter integrated sensors system (or called a safe labor monitor, SLM) specifically designed to monitor maternal conditions, fetal heart rate, and uterine contraction condition (frequency, intensity, propagation velocity, and movement direction) for pregnant women in labor. The sensing slice includes the ultrasound sensor (e.g., a Doppler ultrasonic sensing element) and at least one at least one bioelectrical sensor (e.g., an electrohysterography (EHG) sensing element) based on laser-induced graphene (LIG) materials encapsulated by flexible Polydimethylsiloxane (PDMS) packaging layers. The controller integrates a flexible printed circuit board with a micro-control chip (e.g., the MCU) that incorporates signal amplifier(s), filter(s), converter(s), and Bluetooth, among other components, to enable real-time transmission of signals to the terminal via wireless electronic technology. The terminal encompasses the data processing system. The visual interface unit in the terminal displays various physiological curves of fetal heart patterns, uterine contractions, and health evaluation results to users, e.g., through an artificial intelligence (AI) auxiliary system. The technical solutions in the present disclosure can help to alert the clinicians of risks developing during labor and monitor uterine atony (uterine exhaustion) after delivery to prevent heavy postpartum bleeding. The system may also be designed as a wearable SLM system which is expected to be applied for future precision and personalized, safe labor monitoring.

One or more embodiments provide a novel combination of sensors (e.g., the bioelectrical sensor(s) and the ultrasound sensor) to monitor the uterine labor process and the baby's well-being. It is the inventors' knowledge that no monitoring device is available in the market or ongoing clinical research. It is possible to incorporate other monitoring methods such as Photo Plethysmo Graphic (PPG), Electrocardiogram (ECG), and cuffless blood pressure devices for the mother's well-being monitoring.

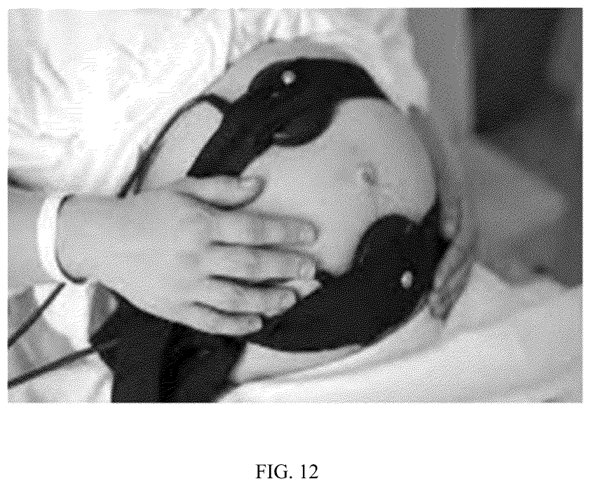

One focal point in one or more embodiments lies in enhancing clinical utility by applying the technical solutions in the present disclosure. The Safe Labor Monitoring (SLM) system introduces a groundbreaking capability to detect uterine contractions' intensity, frequency, direction, and speed, offering a novel and distinctive approach. Notably, one or more embodiments distinguish the disclosed system from conventional, bulky clinical monitoring devices (for example, the cardiotocograph sensors for monitoring the labor process requires belts to fix the sensors to the abdomen, with connecting wires to the cardiotocographic machine, as shown in FIG. 12) by its ultra-thin and flexible nature. Affixed directly to the pregnant woman's abdomen, the SLM system in the present disclosure can ensure ease of use and comfort.

According to one or more embodiments, the SLM system uses an innovative multi-electrode layout in an array of three rows and two columns to improve detection and measurement capabilities. This layout and spacing distance allow the SLM system to accurately monitor the clinical essential characteristics, including strength, frequency, direction, and speed of contractions. In contrast, other existing monitoring systems can only detect the contraction frequency, and thus the present disclosure can maximize the sensor's ability to perceive the contraction. With a carefully designed electrode layout, an extra reference electrode, and sensor fixation, sensitivity can be increased, and external interference is reduced.

One or more embodiments provide a unique combination of materials, including EHG sensing elements based on laser-induced graphene (LIG) materials and Doppler ultrasound energy exchange sensing elements, which have excellent electrical properties and sensitivity, enabling accurate sensing and measurement of contraction signals, thereby increasing the sensitivity of the sensors.

Unlike other designs, the manufacturing approach in the present disclosure encapsulates the sensing elements (i.e., the electrodes) in a flexible PDMS layer in one or more embodiments. This package protects the sensing elements, reduces external interference, and improves sensitivity. It also makes the device flexible so that the sensitivity can be enhanced by lowering rigidity, making it more fit for human skin.

In one or more embodiments, the sensing slice has a SPI component, and the sensing slice and the controller may be detachably connected via SPI components arranged in the sensing slice and the controller, respectively. The SPI component in the sensing slice allows for the seamless interchangeability of the sensing slice (or called a sensor patch), maintaining a fixed circuit part. This feature not only upholds sensor privacy and cleanliness but also contributes to environmental preservation and resource conservation. Leveraging ultrasonic sensing, the SLM system provides a multifaceted input, and the exceptional electrical properties of graphene and the innovative multi-electrode layout markedly heighten its sensitivity.

One or more embodiments position the SLM monitoring as a transformative technology, offering unprecedented capabilities for monitoring uterine contractions in a technologically advanced and user-friendly manner.

An embodiment further provides a method for detecting fetal heart rate and uterine contraction indicators, including frequency, intensity, propagation velocity, and movement direction in pregnant women, includes real-time monitoring using the flexible wireless sensor and analyzing and processing the measured data.

An embodiment provides a safe labor monitoring system for pregnant women, the system includes a novel combination of sensors (bioelectrical sensor and ultrasound sensor) to monitor the uterine labor process and the baby's well-being. The above described safe labor monitoring method can be applied in this system.

An embodiment provides a method of operating the system. An embodiment provides a system of performing the method.

The following describes the functions and applications of the present disclosure.

The flexible device, the system or the method(s), according to one or more embodiments, may facilitate one or more applications, including but not limited to the following:

Scientific discoveries—Scientists and research institutions want to improve safe, real-time labor monitoring. One or more embodiments provide techniques to integrate ultra-thin flexible devices with conformal fetal heart rate and uterine contraction sensors into flexible digitalized electronics and develop machine learning algorithms to monitor maternal and fetal safety during labor.

Labor Monitoring—According to one or more embodiments, the devices, or systems or methods in the present disclosure can arouse pregnant women and clinicians' interest in providing real-time, long-lasting, flexible multi-indicator labor monitoring to ensure maternal and fetal safety in labor. Additionally, the present disclosure provides a low-cost replacement option for traditional cardiotocogram monitoring in labor. This affordable life-saving technology development may become available to women in labor in poor economic areas.

Objectives of clinical evaluation—Clinicians are particularly interested in monitoring fetal heart rate and uterine contraction frequency, intensity, and direction in real-time clinical studies to effectively assess labor's progress and safety. The continuous monitoring of maternal blood pressure and pulses may also indicate the woman's health condition.

Free movement during monitoring: Mothers prefer freedom of movement instead of lying in bed in pain during labor. The wireless transmission of digital data from the small ultrasound and pressure sensor device to a remote display unit (e.g., the visual interface in FIG. 6) will allow total freedom of movement for the woman in labor. Telemedicine applications of the flexible device may become a cost-effective method for detecting early labor before hospital admission. This can help alleviate the demanding pressure on bed allocation and resources in public hospitals.

The labor monitoring system with wireless transmission of digital labor data may help to report premature labor, helping to monitor women at risk of premature labor before 36 weeks, to alert them to arrive hospital earlier, improving the morbidity and mortality of premature babies.

### Further Advantages

In addition to the technical advantages mentioned above, the flexible devices, the systems or methods according to one or more embodiments has, but may not be limited to, one or more of the following technical advantages:

Cost-effective: for example, the pressure sensor electrodes (e.g., the EHG electrodes in FIG. 3) are manufactured using laser engraving, enabling efficient mass production and saving costs.

Flexible sensing capability: the ultra-thin and conformal design in the present disclosure allows the electrodes in the sensing slice to adhere to the skin surface, enhancing user comfort efficiently. The associated wireless electronics (e.g., the Bluetooth module in FIG. 4) are integrated into a soft patch (e.g., the flexible printed circuit board in FIG. 5) that offers improved malleability and adhesion when attached to the skin.

Portability: For example, the flexible device in the present disclosure is lightweight and easy to use, and the flexible device, according to one or more embodiments, is portable and suitable for home use and deployment in public medical facilities or areas with limited resources and nursing capacity.

Multi-mode monitoring: for example, the sensing unit in the flexible device is a multi-sensor unit including a fixed-point ultrasound (US) and electrohysterography (EHG) system with seven channels, enabling analysis of fetal heart rate, and the frequency, intensity, propagation velocity, and the direction of contraction forces are computed and analyzed. The multi-sensor unit ensures the stability of the signal and can detect abnormal signals with high precision. Signal transmission utilizes a non-balanced differential electrode multiplexing method to enhance signal acquisition bandwidth and reduce artifacts caused by electrode drift and amplifiers. By simplifying the complexity of the sensor client, interference around the signal collector is minimized.

Intelligent diagnosis: for example, leveraging machine learning techniques, the present disclosure can classify subjects' contraction levels based on multi-indicator analysis, predict labor time labor progress, and assess the health status of mother and baby. Timely screening for invalid contractions or abnormal fetal position can predict and prevent abnormal labor and fetal outcomes.

Cleanliness and privacy: for example, the flexible sensing slice (or called a flexible electrode patch) can be easily disassembled and replaced at a low cost, making it suitable for use in public healthcare facilities while ensuring the privacy of the monitoring process.

Multi-time dimension monitoring: for example, the SLM system can be used to monitor the safety of the mother and the fetus during delivery through the EHG signals array, to monitor uterine tension (uterine failure) after delivery, to monitor consistent uterine contraction pressure, and prevent postpartum bleeding due to uterine atony.

In addition, compared to existing monitoring system(s), this sensor model provided in the present disclosure is economical, affordable in many countries, and environmentally friendly, and the remote monitoring capacity can allow more widespread use in isolated environments at home or during a viral pandemic.

As one example, compared to the existing cardiotocogram (as shown in FIG. 12), the product model is light, allowing mobilization and remote monitoring of the well-being of the mother and baby in labor. Research studies of the labor process can be practical at home, clinic, or hospital. The system(s), device(s) or method(s) in the present disclosure may be applied to clinical practice.

It should be noted that although some sizes or dimensions are labeled in drawings, such sizes or dimensions are only illustrative, and should not be construed as constituting any limitation on the present disclosure. Further, the figures are not necessarily drawn to scale, and for convenience of description, some parts in the figures may be illustrated in an exaggerated manner.

The foregoing descriptions are merely example embodiments of the present disclosure, but the protection scope of the present disclosure is not limited thereto. Any person skilled in the art can easily think of changes or substitutions within the technical scope of the present disclosure, and all the changes or substitutions should be covered by the protection scope of the present disclosure. Therefore, the protection scope of the present disclosure should be defined by the appended claims.

What is claimed is:

1. A flexible device, comprising a flexible sensing slice, wherein the flexible sensing slice comprises:
a combination of an ultrasound sensor and at least one bioelectrical sensor, wherein the at least one bioelectrical sensor is fabricated by processing a polyimide film using a laser with Laser-Induced Graphene (LIG) as a sensing material, and the ultrasound sensor is placed on the polyimide film; and
a Polydimethylsiloxane (PDMS) film encapsulating the combination of the ultrasound sensor and the at least one bioelectrical sensor.

2. The flexible device according to claim 1, wherein the at least one bioelectrical sensor comprises multiple bioelectrical sensors in an array of rows and columns for measuring uterine contraction indexes;
wherein the ultrasound sensor comprises an ultrasonic transducer configured to measure a fetal heart rate.

3. The flexible device according to claim 2, wherein the multiple bioelectrical sensors comprise six electrohysterography (EHG) electrodes arranged in three rows and two columns for EHG signal reading and one reference EHG electrode for reference measurement;
wherein the one reference EHG electrode is arranged between two columns of the six EHG electrodes.

4. The flexible device according to claim 1, further comprising:
a controller configured to receive and process signals of the ultrasound sensor and the at least one bioelectrical sensor, wherein the controller comprises:
a flexible printed circuit board;
a microcontroller unit (MCU) formed on the flexible printed circuit board; and
top and bottom PDMS films encapsulating the flexible printed circuit board.

5. The flexible device according to claim 4, wherein the flexible sensing slice further comprises a first Serial Peripheral Interface (SPI) component connected with the ultrasound sensor and the at least one bioelectrical sensor;
wherein the controller comprises: a second SPI component detachably connected with the first SPI component.

6. The flexible device according to claim 5, wherein the controller further comprises:
a transceiver configured to transmit measurement data of the ultrasound sensor and the at least one bioelectrical sensor to a terminal wirelessly; and
a flexible battery connected with the second SPI component to provide power supply for the flexible sensing slice;
wherein the transceiver and the flexible battery are printed on the flexible printed circuit board.

7. The flexible device according to claim 3, wherein each of the six EHG electrodes and the one reference EHG electrode has a ring shape.

8. The flexible device according to claim 3, wherein the two columns are arranged symmetrically with respect to a virtual line for connecting a center of the one reference EHG electrode and a center of the ultrasound electrode; and
wherein vertical spacing between adjacent EHG electrodes among the six EHG electrodes is equal.

9. A method for manufacturing a flexible device, comprising:
fabricating at least one bioelectrical sensor in a flexible sensing slice of the flexible device by processing a polyimide film using a laser with Laser-Induced Graphene (LIG) as a sensing material, and reserving a space for an ultrasound sensor in the flexible sensing slice of the flexible device;
placing the ultrasound sensor in the space; and
placing a PDMS film to encapsulate the combination of the ultrasound sensor and the at least one bioelectrical sensor.

10. The method according to claim 9, wherein before placing the ultrasound sensor in the space, the method further comprises:
pouring PDMS liquid on the polyimide film on which a pattern of the at least one bioelectrical sensor has been completed;
exposing the polyimide film with the PDMS liquid poured;
removing a PDMS layer from the polyimide film after being heated by a hot drying plate.

11. The method according to claim 9, further comprising:
forming a flexible printed circuit board of a controller of the flexible device, wherein a microcontroller unit (MCU) is formed on the flexible printed circuit board; and
placing top and bottom PDMS films to encapsulate the flexible printed circuit board.

12. The method according to claim 11, wherein forming the flexible printed circuit board comprises:
coating a conductive material on a flexible substrate;
applying a photoresist material on the flexible substrate, performing exposure to ultraviolet (UV) light through a photomask containing a desired circuit pattern;
developing exposed photoresist material, and leaving a patterned photoresist resist layer that protects underlaying conductive traces formed from the conductive material; and
attaching the MCU to the flexible substrate; and
coating a protective layer on the flexible substrate.

13. The method according to claim 12, further comprising:
attaching a second Serial Peripheral Interface (SPI) component to the flexible substrate.

14. The method according to claim 13, further comprising:
attaching a transceiver and a flexible battery to the flexible substrate,
wherein transceiver is configured to transmit measurement data of the ultrasound sensor and the at least one bioelectrical sensor to a terminal wirelessly, and the flexible battery is connected with the second SPI component to provide power supply for the flexible sensing slice.

15. A monitoring system comprising:
a flexible device, comprising a flexible sensing slice, wherein the flexible sensing slice comprises:
a combination of an ultrasound sensor and at least one bioelectrical sensor, wherein the at least one bioelectrical sensor is fabricated by processing a polyimide film using a laser with Laser-Induced Graphene (LIG) as a sensing material, and the ultrasound sensor is placed on the polyimide film; and
a Polydimethylsiloxane (PDMS) film encapsulating the combination of the ultrasound sensor and the at least one bioelectrical sensor; and
a terminal configured to receive measurement data of the ultrasound sensor and the at least one bioelectrical sensor wirelessly.

16. The system according to claim 15, wherein the flexible device further comprises:
a controller configured to receive and process signals of the ultrasound sensor and the at least one bioelectrical sensor, wherein the controller comprises:
a flexible printed circuit board;
a microcontroller unit (MCU) formed on the flexible printed circuit board; and
top and bottom PDMS films encapsulating the flexible printed circuit board.

17. The system according to claim 16, wherein the controller further comprises:
a transceiver configured to transmit measurement data of the ultrasound sensor and the at least one bioelectrical sensor to the terminal wirelessly; and
a flexible battery connected with a second SPI component in the controller to provide power supply for the flexible sensing slice;
wherein the transceiver and the flexible battery are printed on the flexible printed circuit board.

18. The system according to claim 17, wherein the terminal comprises:
a data processing system configured to receive and analyze data from the controller; and
a visual interface configured to display physiological curves of fetal heart patterns, or uterine contractions based on an analysis result of the data processing system.

19. The system according to claim 18, further comprising:
a cloud server configured to upload data to a cloud for data storage and analysis.

20. The system according to claim 18, further comprising:
an alarm configured to send an alarm signal when an abnormal situation is detected based on analysis of data processing system.
